(19) United States
(12) Patent Application Publication
(10) Pub. No.: US 2012/0143172 A1
(43) Pub. Date: Jun. 7, 2012

(54) Assembly For Use With Surgery System

(75) Inventors: Thomas P. Oko, Shelton, CT (US); Jason K. Blake, Orange, CT (US)

(73) Assignee: ALOKA COMPANY, LTD.

(21) Appl. No.: 12/958,953

(22) Filed: Dec. 2, 2010

(51) Int. Cl. A61B 17/00

(52) U.S. Cl. 606/1

(57) ABSTRACT

The present disclosure provides systems, assemblies and methods for surgery (e.g., robotic surgery). More particularly, the present disclosure provides systems and methods for releasably securing or attaching an assembly for use in a surgical procedure with respect to a user-operable surgical device. In general, the present disclosure provides systems and methods for releasably securing or attaching an assembly with respect to and for use with a user-operable surgery system. Systems and methods for releasably securing or attaching an assembly (e.g., imaging assembly) having a receiver member with respect to a user-operable grasper member of a surgery system are provided.

### BACKGROUND

1. Technical Field

The present disclosure relates to advantageous systems, assemblies and methods for surgery (e.g., robotic surgery) and, more particularly, to a system and method for releasably securing or attaching an assembly (e.g., a surgical or imaging assembly having a receiver member) with respect to a surgical device (e.g., with respect to a grasper member of a surgery system).

2. Background Art

Minimally invasive surgical systems or the like are known. Minimally invasive surgery typically presents some advantages compared to traditional and/or open surgery procedures (e.g., reduced scarring and/or recovery time, decreased injury/pain to the patient, decreased hospitalization time, etc.). Minimally invasive surgery is generally known under various names (e.g, endoscopy, laparoscopy, arthroscopy, etc.), with the names typically being specific to the anatomical area of the surgery. For example, laparoscopic surgery, which is one type of minimally invasive surgery, is a more recent surgical technique where operations in the abdomen are performed through small incisions (e.g., about 1.0 cm) as compared to larger incisions typically required in traditional surgical procedures.

In general, telesurgery systems allow a surgeon to operate on a patient from a remote location. Telesurgery is a general term for surgical systems (e.g., robotic surgical systems) where the surgeon uses some form of servo-mechanism to manipulate the surgical instruments movements rather than directly holding and moving some of the tools. Robotic surgical systems such as minimally invasive robotic surgical systems or the like are generally known. Telesurgery systems have been utilized for both open and endoscopic procedures.

During a minimally invasive surgical procedure (robotic or manual), tubes or the like (e.g., cannulas or trocars or other tool guides) may be inserted through the same or different incisions so that assemblies, devices, probes and/or surgical instruments/tools may be introduced to the desired surgical site. In general, many different surgical procedures can then be performed without requiring a large and/or open cavity incision as typically required by traditional surgical procedures. The laparoscopic surgical instruments generally are similar to those used in conventional (open) surgery, except that the working end of each tool is separated from its handle by an approximately 12-inch long extension tube. The surgeon (and/or robotic system) typically passes instruments through a cannula or the like and manipulates them inside the abdomen by sliding them in and out through the cannula, rotating them in the cannula, levering or pivoting the instruments in the abdominal wall and actuating end effectors on the distal end of the instruments.

In general, imaging devices or the like (e.g., ultrasound probes/transducers and/or assemblies, endoscopes, cameras, etc.) and other surgical instruments/assemblies (e.g., clamp members/instruments, grasper members/instruments, blades, needles, scissors, holder members/instruments, staplers, etc.) for use with minimally invasive surgical systems (e.g., robotic or manual) are known. For example, imaging devices such as ultrasound assemblies and/or probes or the like that are introduced to the desired surgical site provide images of the site to the surgeon. As noted, minimally invasive tools or devices are typically configured and dimensioned to be inserted through a cannula or trocar or other tool guide located in a minimally invasive incision of the patient in order to extend the surgical tools or devices to the surgical site. Exemplary minimally invasive robotic surgical systems are disclosed, for example, in U.S. Pat. Nos. 5,797,900; 5,876,325; 6,371,952 and 7,107,090; and U.S. Patent Publication Nos. 2007/0021738; 2008/0064921; 2009/0088773; 2009/0192519; 2009/0245600; 2009/0248041 and 2009/0326318; the foregoing being incorporated herein by reference in their entireties.

In general and as disclosed in the above listed references, robotic surgical systems typically include user-operable master input devices (e.g., joysticks, gloves, trigger-guns, hand-operated controllers, etc.) that allow a user to manipulate them to have a processor then cause their respectively associated slave arms or the like manipulate their respectively coupled and/or held surgical instruments and/or devices. In short, a surgeon typically performs a minimally invasive surgical procedure with a robotic system by manipulating the master input devices to control (via a processor) the robotic slave arms, which have tools, instruments, probes, etc. attached thereto. Robotic surgical systems typically also include a master display or display screen. Current practice provides that surgeons or technicians are frequently confronted with the need to move, position, re-position, align and/or adjust various assemblies/devices/tools or the like (e.g., surgical assemblies, imaging assemblies, ultrasound probes/transducers, endoscopes, blades, etc.) during surgery (robotic or manual surgery) under difficult conditions (e.g., in confined/tight spaces, in conjunction with robotic surgical systems, etc.). Such movements/procedures can be very difficult and/or time consuming, especially when the surgical and/or imaging assemblies or the like are associated with and/or utilized along with minimally invasive surgical systems (e.g., minimally invasive robotic surgical systems).

With the foregoing in mind, those skilled in the art will understand that a need exists to provide a patient with an assembly for use in a surgical procedure that is capable of releasably securing or attaching to a user-operable surgical device (e.g., a user-operable surgical device associated with a minimally invasive surgery system, such as a robotic surgery system). Thus, despite efforts to date, a need remains for improved and efficient systems/methods for releasably securing or attaching an assembly for use in a surgical procedure (e.g., an imaging assembly having a receiver member) with respect to a surgical device (e.g., with respect to and for use with a grasper member of a robotic surgery system).

These and other challenges and opportunities for improvement are addressed and/or overcome by the systems, assemblies and methods of the present disclosure.

### SUMMARY

The present disclosure provides advantageous systems, assemblies and methods for surgery (e.g., robotic surgery). More particularly, the present disclosure provides improved systems and methods for releasably securing or attaching an assembly for use in a surgical procedure with respect to a user-operable surgical device. In general, the present disclosure provides improved systems and methods for releasably attaching or securing an assembly (e.g., a surgical or imaging assembly) with respect to and for use with a user-operable robotic surgery system. In exemplary embodiments, the present disclosure provides advantageous systems and methods for releasably securing or attaching an assembly having a receiver member with respect to a user-operable grasper member of a robotic or manual surgery system.

The present disclosure provides for an assembly for use in a surgical procedure including a housing defining a substantially fin-shaped receiver member, the receiver member including a post member and a securing member; wherein the post member extends from the housing and the securing member extends past both sides of the post member to define the substantially fin-shaped receiver member; and wherein the substantially fin-shaped receiver member is configured and dimensioned to be releasably secured to a user-operable surgical device.

The present disclosure also provides for an assembly for use in a surgical procedure further including an imaging member mounted with respect to the housing. The present disclosure also provides for an assembly for use in a surgical procedure wherein the imaging member is an ultrasound transducer. The present disclosure also provides for an assembly for use in a surgical procedure wherein the receiver member is positioned at or near the proximal end of the housing.

The present disclosure also provides for an assembly for use in a surgical procedure wherein the user-operable surgical device includes first and second end effectors, the first end effector having a first slit and the second end effector having a second slit; wherein the securing member has a first side and a second side; and wherein at least a portion of the first side of the securing member extends through the first slit and at least a portion of the second side of the securing member extends through the second slit when the user-operable surgical device is releasable secured to the receiver member.

The present disclosure also provides for an assembly for use in a surgical procedure wherein the receiver member is integrally formed from the housing. The present disclosure also provides for an assembly for use in a surgical procedure wherein the securing member has a first end and a second end and the post member defines a longitudinal axis; and wherein the first end of the securing member extends a greater distance from the longitudinal axis relative to distance that the second end of the securing member extends from the longitudinal axis. The present disclosure also provides for an assembly for use in a surgical procedure wherein the post member has a first end and a second end, the first end wider than the second end.

The present disclosure also provides for an assembly for use in a surgical procedure wherein at least a portion of the receiver member is retractable within the housing. The present disclosure also provides for an assembly for use in a surgical procedure wherein at least a portion of the receiver member is foldable with respect to the housing. The present disclosure also provides for an assembly for use in a surgical procedure wherein the housing is mounted with respect to a flexible cable. The present disclosure also provides for an assembly for use in a surgical procedure wherein the user-operable surgical device is a minimally invasive user-operable surgical device. The present disclosure also provides for an assembly for use in a surgical procedure wherein the imaging member is configured and dimensioned to capture an image of a surgical site.

The present disclosure also provides for an assembly for use in a surgical procedure wherein the housing and receiver member are configured and dimensioned to be: (i) inserted through a guide tool located in a minimally invasive incision of a patient, and (ii) moved to a surgical site within the patient.

The present disclosure also provides for a method for attaching an assembly to a surgical device including providing a housing defining a substantially fin-shaped receiver member, the receiver member including a post member and a securing member, with the post member extending from the housing and the securing member extending past both sides of the post member to define the substantially fin-shaped receiver member; and releasably securing a user-operable surgical device to the substantially fin-shaped receiver member.

The present disclosure also provides for a method for attaching an assembly to a surgical device further including mounting an imaging member with respect to the housing. The present disclosure also provides for a method for attaching an assembly to a surgical device wherein the imaging member is an ultrasound transducer. The present disclosure also provides for a method for attaching an assembly to a surgical device wherein the user-operable surgical device includes first and second end effectors, the first end effector having a first slit and the second end effector having a second slit; wherein the securing member has a first side and a second side; and wherein at least a portion of the first side of the securing member extends through the first slit and at least a portion of the second side of the securing member extends through the second slit when the user-operable surgical device is releasable secured to the receiver member.

The present disclosure also provides for a method for attaching an assembly to a surgical device wherein the securing member has a first end and a second end and the post member defines a longitudinal axis; and wherein the first end of the securing member extends a greater distance from the longitudinal axis relative to distance that the second end of the securing member extends from the longitudinal axis.

The present disclosure also provides for a method for attaching an assembly to a surgical device wherein the post member has a first end and a second end, the first end wider than the second end. The present disclosure also provides for a method for attaching an assembly to a surgical device wherein at least a portion of the receiver member is retractable within the housing.

The present disclosure also provides for an imaging assembly including a housing defining a substantially fin-shaped receiver member, the receiver member including a post member and a securing member; an imaging member mounted with respect to the housing; wherein post member extends from the housing and the securing member extends past both sides of the post member to define the substantially fin-shaped receiver member; wherein the securing member has a first end and a second end and the post member defines a longitudinal axis; wherein the first end of the securing member extends a greater distance from the longitudinal axis relative to distance that the second end of the securing member extends from the longitudinal axis; wherein the receiver member is positioned at or near the proximal end of the housing; wherein the substantially fin-shaped receiver member is configured and dimensioned to be releasably secured to a user-operable surgical device; wherein the user-operable surgical device includes first and second end effectors, the first end effector having a first slit and the second end effector having a second slit; wherein the securing member has a first side and a second side; and wherein at least a portion of the first side of the securing member extends through the first slit and at least a portion of the second side of the securing member extends through the second slit when the user-operable surgical device is releasable secured to the receiver member.

Additional advantageous features, functions and applications of the disclosed systems, assemblies and methods of the present disclosure will be apparent from the description which follows, particularly when read in conjunction with the appended figures.

### BRIEF DESCRIPTION OF THE DRAWINGS

To assist those of ordinary skill in the art in making and using the disclosed systems, assemblies and methods, reference is made to the appended figures, wherein:

FIG. 1 is a side perspective view of an exemplary assembly for use in a surgical procedure in accordance with the present disclosure;

FIG. 2 is a side perspective view of the assembly of FIG. 1;

FIG. 3 is a side view of the assembly of FIG. 1;

FIG. 4 is a partial side perspective view of the assembly of FIG. 1, with the receiver member unattached;

FIG. 5 is a bottom view of the receiver member of the assembly of FIG. 1;

FIG. 6 is a side view of the receiver member of FIG. 5;

FIG. 7 is a proximal end view of the receiver member of FIG. 5;

FIG. 8 is a distal end view of the receiver member of FIG. 5;

FIG. 9 is an in situ side perspective view of an exemplary user-operable surgical device for use with the exemplary assembly of FIG. 1;

FIGS. 10-11 are side perspective views of the assembly of FIG. 1 with the user-operable surgical device of FIG. 9 prior to attachment thereto;

FIG. 12 is a partial side perspective view of the assembly of FIG. 1 with the user-operable surgical device of FIG. 9 attached thereto;

FIG. 13 is an in situ side perspective view of the assembly of FIG. 1 with the user-operable surgical device of FIG. 9 attached thereto;

FIG. 14 is a partial sectional side perspective view of an alternative embodiment of an assembly for use in a surgical procedure according to the present disclosure, the assembly positioned within a tool guide;

FIG. 15 is an exploded partial sectional side view of the assembly of FIG. 14, the receiver member of the assembly in the retracted position;

FIG. 16 is an exploded partial sectional side view of the assembly of FIG. 14, the assembly positioned at least partially out of the tool guide, the receiver member of the assembly in the un-retracted position;

FIG. 17 is a partial side perspective view of an alternative embodiment of an assembly for use in a surgical procedure according to the present disclosure, the receiver member of the assembly in the folded position;

FIG. 18 is a partial side perspective view of the assembly of FIG. 17, the receiver member of the assembly in the un-folded position;

FIG. 19 is a partial sectional side view of an alternative embodiment of an assembly for use in a surgical procedure according to the present disclosure, the receiver member of the assembly in the retracted position; and

FIG. 20 is a partial sectional side view of the assembly of FIG. 19, the receiver member of the assembly in the un-retracted position.

### DESCRIPTION OF EXEMPLARY EMBODIMENTS

The exemplary embodiments disclosed herein are illustrative of advantageous assemblies (e.g., imaging or surgical assemblies) for use with surgery systems and methods/techniques thereof. It should be understood, however, that the disclosed embodiments are merely exemplary of the present disclosure, which may be embodied in various forms. Therefore, details disclosed herein with reference to exemplary assemblies/systems and associated methods/techniques of assembly and use are not to be interpreted as limiting, but merely as the basis for teaching one skilled in the art how to make and use the advantageous assemblies/systems and/or alternative surgical and/or imaging devices of the present disclosure.

The present disclosure provides improved systems, assemblies and methods for surgery (e.g., robotic surgery). More particularly, the present disclosure provides advantageous systems and methods for releasably attaching or securing an assembly (e.g., an imaging or surgical assembly) with respect to a user-operable surgical device. In general, the present disclosure provides systems and methods for releasably attaching or securing an assembly for use in a surgical procedure with respect to and for use with a user-operable robotic surgery system. In exemplary embodiments, the present disclosure provides advantageous systems and methods for releasably attaching or securing an assembly having a receiver member with respect to a user-operable grasper member of a robotic or manual surgery system.

In exemplary embodiments, the assembly for use in a surgical procedure includes at least one receiver member, the at least one receiver member configured and dimensioned to be releasably secured to a user-operable surgical device (e.g., a user-operable grasper member of a robotic or manual surgery system). In general, the assembly for use in a surgical procedure includes an imaging member (e.g., ultrasound probe/transducer, endoscope, camera, etc.) and/or a surgical instrument/tool/device (e.g., clamp members/instruments, blades, needles, scissors, holder members, staplers, etc.) or the like, and/or some other treatment instrument/device.

In exemplary embodiments, the assembly for use in a surgical procedure includes at least one receiver member, component or protrusion (e.g., a T-shaped or fin-shaped protrusion) that allows the assembly to be releasably secured with respect to a user-operable surgical device. A user may then manipulate the user-operable surgical device to move/position the releasably secured surgical or imaging assembly to any desired position and/or location. For example, the assembly may include a T-shaped or fin-shaped protrusion at one end that extends from the assembly to allow the user operable surgical device to releasably secure or attach to at least a portion of the protrusion of the assembly. The receiver member may define at least one cavity, recess, channel or receiving feature that allows the user-operable surgical device to releasably secure to the surgical or imaging assembly.

Current practice provides that it is often very difficult and/or time consuming for a surgeon or technician to move, position, re-position, align and/or adjust assemblies or the like (e.g., surgical or imaging assemblies) during surgery, especially when the assemblies or the like are associated with and/or utilized along with minimally invasive surgical systems (e.g., minimally invasive robotic surgical systems). In exemplary embodiments, the present disclosure provides for improved and effective systems/designs for assemblies that are easily releasably attached or secured to a user-operable surgical device (e.g., a user-operable surgical device associated with a minimally invasive surgery system, such as a robotic surgery system), thereby providing a significant manufacturing, commercial and surgical advantage as a result. Furthermore, the exemplary assemblies/systems may also be capable of attaching, mounting and/or mating with respect to other user-operable surgical devices, thereby providing a significant manufacturing, commercial and surgical advantage as a result.

Referring now to the drawings, like parts are marked throughout the specification and drawings with the same reference numerals, respectively. Drawing figures are not necessarily to scale and in certain views, parts may have been exaggerated for purposes of clarity.

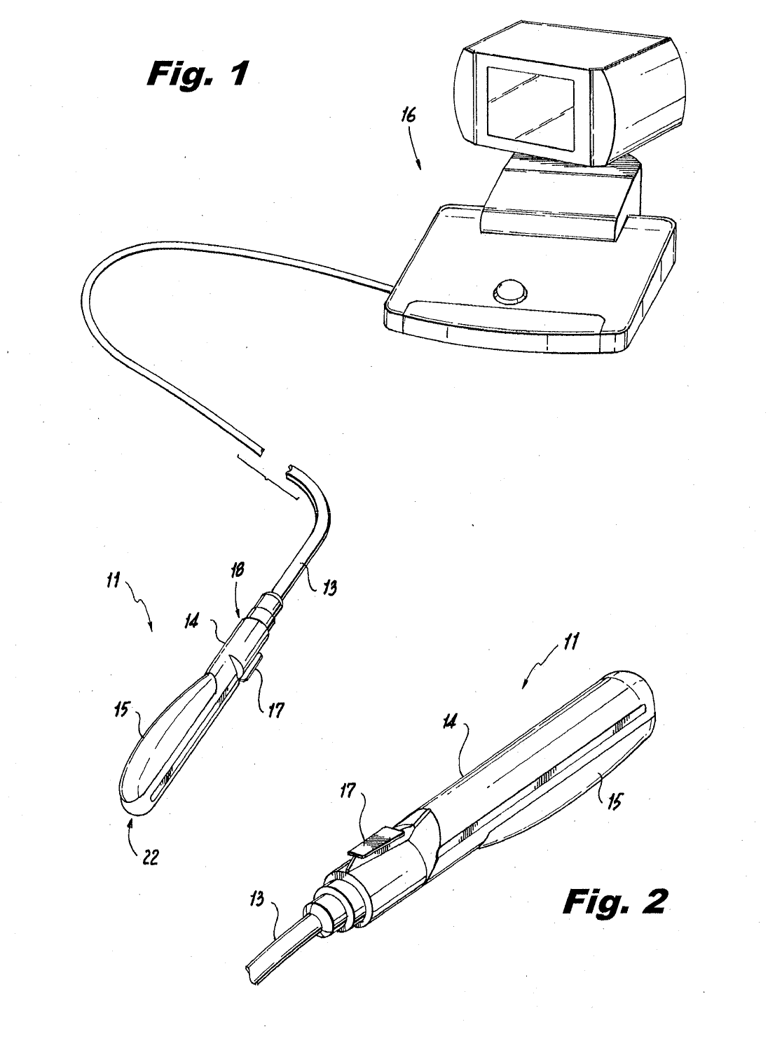

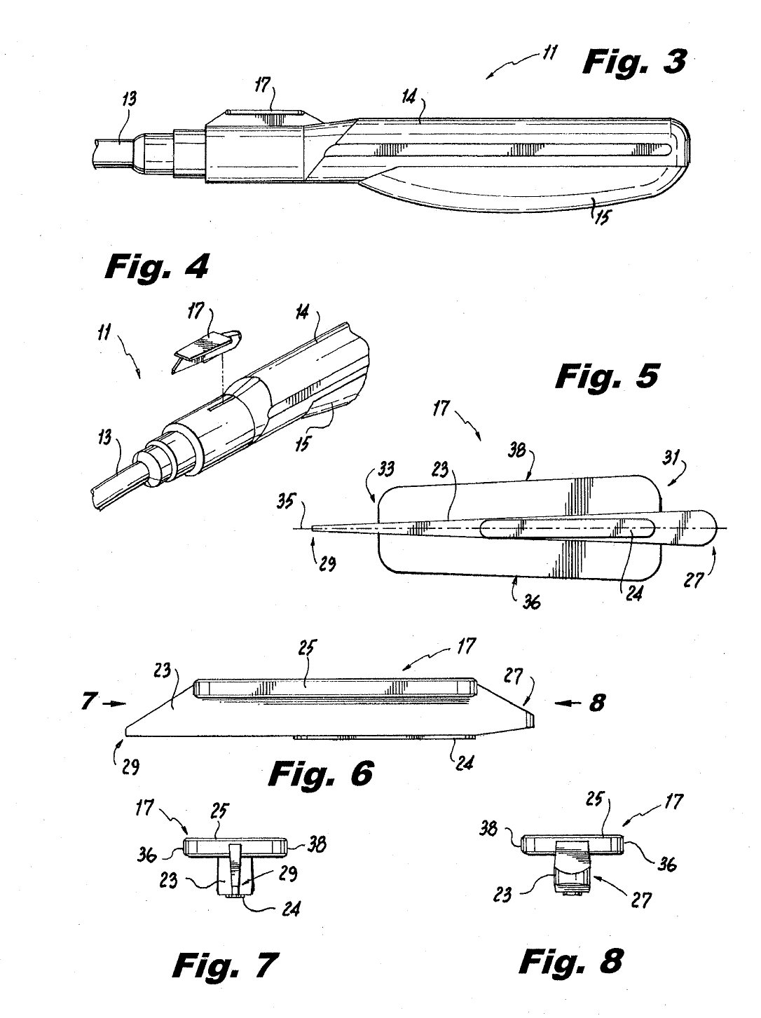

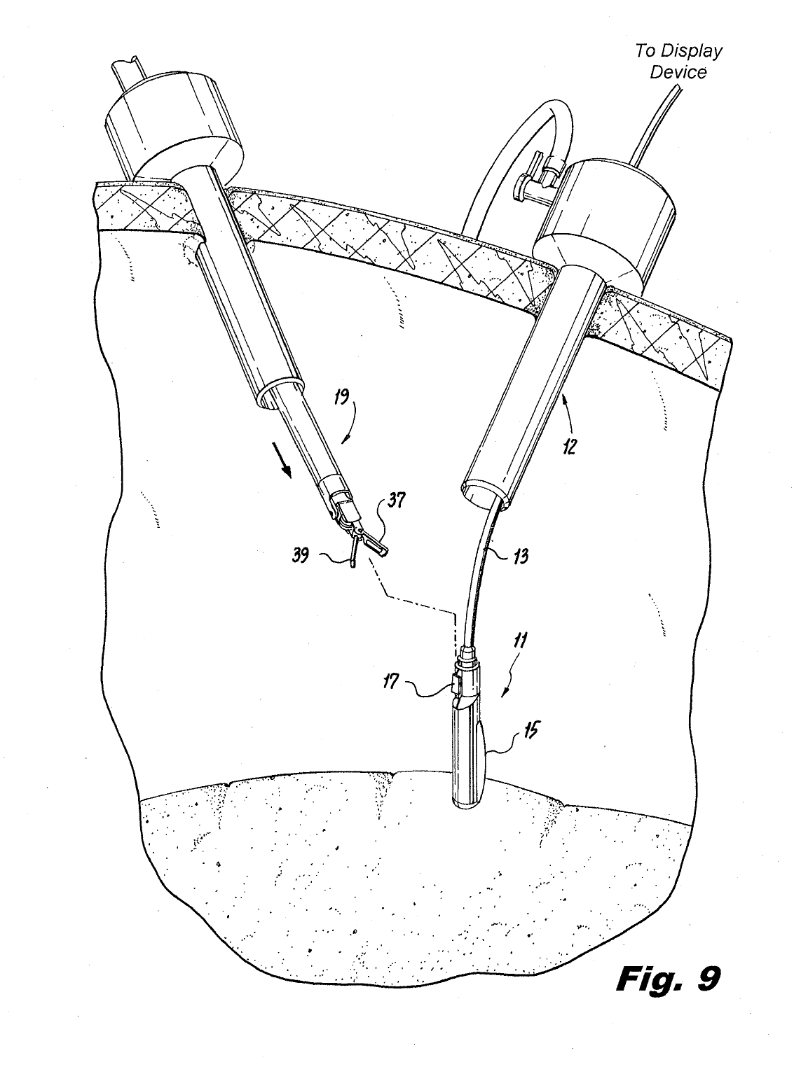

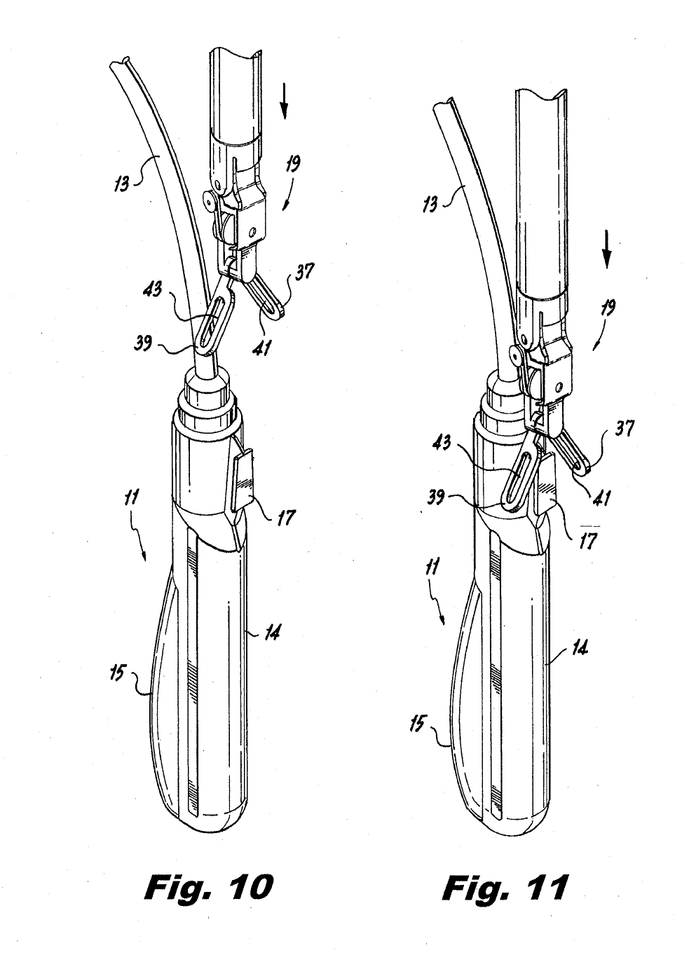

FIGS. 1-20 depict exemplary embodiments of the advantageous assemblies, systems and methods of the present disclosure. As shown in FIGS. 1-4, an exemplary assembly 11 for use in a surgical procedure is typically attached or mounted with respect to a flexible cable 13 or the like. In general, cable 13 allows assembly 11 to be introduced to a surgical site (e.g., in a minimally invasive manner) for imaging and/or surgical purposes or the like (see, e.g., FIGS. 9 and 13). For example, assembly 11 with respective cable 13 attached thereto is typically configured and dimensioned to be inserted through a cannula or trocar or other tool guide 12 (FIG. 9) located in a minimally invasive incision of a patient in order to allow a surgeon or technician to extend at least a portion of the assembly 11 to a surgical site for imaging and/or surgical purposes. Assembly 11 may also be inserted through a body orifice, or utilized in other surgical procedures (e.g., open surgery).

In exemplary embodiments, assembly 11 is connected to and/or in communication with a display device/assembly 16 for displaying images of the surgical site, the display device 16 generally in communication with a processor and being positioned outside of the body of the patient. In one embodiment, cable 13 connects imaging assembly 11 to display device 16 for displaying captured images of the surgical site. However, other variations and modifications are possible. It is noted that assembly 11 is to be construed broadly to include image capture components or members and their associated wiring, cabling, circuitry, hardware and/or display devices. Assembly 11 may relay image data via wired or wireless connections to display device 16 (e.g., to display device 16 positioned outside of the body of the patient).

Exemplary assembly 11 takes the form of an imaging assembly (e.g., an ultrasound imaging assembly), although the present disclosure is not limited thereto. Rather, assembly 11 may take a variety of forms, including without limitation an endoscopic imaging assembly, an optical imaging assembly, an infrared imaging assembly, a camera-based imaging assembly or the like. It is noted that assembly 11 may or may not include an imaging member or the like. For example, assembly 11 may include a surgical member/instrument/tool/device (e.g., clamp members/instruments, blades, needles, scissors, holder members, staplers, grasper members, etc.) or the like, and/or some other treatment member/instrument/device (e.g., for use in a surgical procedure).

As shown in FIGS. 1-4, exemplary assembly 11 for use in a surgical procedure includes housing 14. In one embodiment, assembly 11 takes the form of an imaging assembly 11 (e.g., an ultrasound imaging assembly), although the present disclosure is not limited thereto. Rather and as noted above, assembly 11 for use in a surgical procedure may take a variety of forms. Assembly 11 is typically configured and dimensioned to be inserted through a cannula or trocar or other tool guide 12 located in a minimally invasive incision of a patient in order to allow a surgeon or technician to extend the assembly 11 to a surgical site for imaging and/or surgical/treatment purposes.

In exemplary embodiments, housing 14 is configured and dimensioned to house, secure and/or mount with respect to an imaging member 15. Exemplary imaging member 15 takes the form of an ultrasound transducer, although the present disclosure is not limited thereto. Rather, imaging member 15 may take a variety of forms (e.g., endoscope, camera, etc.). In general, imaging member 15 is configured and dimensioned to capture images of the surgical site.

In exemplary embodiments, imaging member 15 is an ultrasound transducer that includes a plurality of ultrasonic energy generation elements. As shown in FIG. 1, ultrasound transducer 15 typically extends to the distal end 22 of the housing 14 of ultrasound imaging assembly 11. However and as noted above, the present disclosure is not to be limited to an ultrasonic imaging device/assembly. In exemplary embodiments, ultrasound transducer 15 is configured and dimensioned to obtain two-dimensional or three-dimensional images of the desired surgical site (e.g., in a minimally invasive manner). For example, assembly 11 with ultrasound transducer 15 is typically configured and dimensioned to be inserted through a cannula or trocar or other tool guide 12 located in a minimally invasive incision of a patient in order to allow a surgeon or technician to extend the ultrasound transducer 15 to a surgical site so that the assembly 11 can relay captured ultrasound image data to outside the patient body. Assembly 11 may also be used in other surgical procedures, e.g., open surgery procedures, for imaging and/or surgical purposes.

As depicted in FIGS. 1-13, exemplary assembly 11 (e.g., ultrasound imaging assembly) also includes a receiver member 17. In one embodiment, the housing 14 and the receiver member 17 are of unitary construction with respect to each other (e.g., the receiver member 17 is integrally formed from the housing 14), although the present disclosure is not limited thereto. Alternatively, receiver member 17 may be separately fabricated and then secured, attached or mounted with respect to (e.g., welded) housing 14 (FIG. 4).

In general, receiver member 17 is configured and dimensioned to be releasably secured or attached to a user-operable surgical device 19 (e.g., a user-operable grasper member of a robotic or manual surgery system), as further discussed below in conjunction with FIGS. 9-13. As such, a user may then manipulate the user-operable surgical device 19 to move/position the releasably secured imaging assembly 11 to any desired position and/or location (e.g., in a minimally invasive manner within the surgical site for imaging, surgical and/or diagnostic purposes). In exemplary embodiments, the receiver member 17 is a substantially T-shaped or fin-shaped component or protrusion that extends from the housing 14 (e.g., from or near the proximal end 18 of housing 14) to allow the user operable surgical device 19 to releasably secure or attach to at least a portion of the receiver member 17. The receiver member 17 may also define at least one cavity, recess, channel or receiving feature that allows the user-operable surgical device 19 to releasably secure to the assembly 11.

In exemplary embodiments and as best shown in FIGS. 5-8, receiver member 17 includes a post member 23 that extends from housing 14, and a securing member 25 that extends beyond or past both sides of the post member 23 to define a substantially T-shaped or fin-shaped component or protrusion (i.e., receiver member 17) that extends from the housing 14 (e.g., extends from at or near the proximal end 18 of housing 14). In general, the post member 23 and the securing member 25 are of unitary construction with respect to each other, although the present disclosure is not limited thereto. Post member 23 may also include an attachment member 24, the attachment member 24 being configured and dimensioned to be attached, secured or mounted with respect to the housing 14 (e.g., with respect to a groove or slot of housing 14) of the assembly 11.

The post member 23 has a first end 27 and a second end 29, with the first end 27 typically being wider (e.g., laterally wider) than the second end 29 (FIG. 5). The securing member 25 has a first end 31 and a second end 33, with the first end 31 typically extending (e.g., laterally) a greater distance beyond the longitudinal axis 35 of the post member 23 relative to the extension of the second end 33 of the securing member 25 beyond axis 35 (FIG. 5).

As noted above, assembly 11 may be utilized in conjunction with a user-operable surgical device 19 (FIGS. 9-13), such as, for example, a user-operable grasper member of a robotic or manual surgery system (e.g., a minimally invasive surgery system). For example, a technician or surgeon can operate/move user-operable surgical device 19 either manually (e.g., by operating a conventional laparoscopic surgical device 19) or by robotic tele-surgery operation (e.g., utilizing a robotic surgery system such as a minimally invasive robotic surgery system) within or near the surgical site to releasably secure or attach the user-operable surgical device 19 to assembly 11. Once the user-operable surgical device 19 is releasably secured or attached to the assembly 11, a user may then move/position (e.g., manually or tele-surgically) the assembly 11 to any desired position and/or location (e.g., in a minimally invasive manner within the surgical site for imaging, surgical and/or diagnostic purposes).

As noted above, exemplary robotic surgical systems (e.g., minimally invasive robotic surgical systems) and their operations/movements thereof are disclosed and described in U.S. Pat. Nos. 5,797,900; 5,876,325; 6,371,952 and 7,107,090; and U.S. Patent Publication Nos. 2007/0021738; 2008/0064921; 2009/0088773; 2009/0192519; 2009/0245600; 2009/0248041 and 2009/0326318; the entire contents of each being hereby incorporated by reference in their entireties.

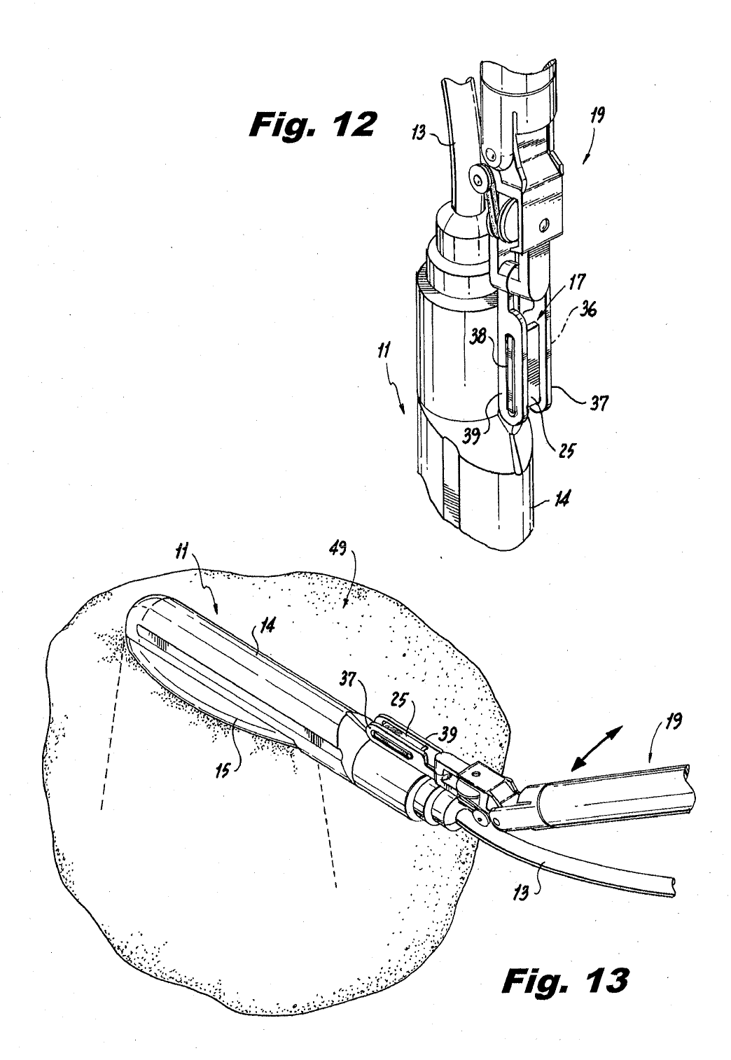

In one embodiment, the user-operable grasper member 19 includes first and second end effectors 37, 39 (e.g., first and second jaws or grasping members 37, 39), with each end effector 37, 39 having respective slots 41, 43. As such, a user may operate the user-operable grasper member 19 (e.g., either manually or tele-surgically) to firstly open or widen the first and second end effectors 37, 39, and then secondly to position the slots 41, 43 of grasper member 19 adjacent to the left side and right side 36, 38 of securing member 25, respectively. The user may then operate the grasper member 19 to then close the end effectors 37, 39 so that at least a portion of the left side 36 of securing member 25 is releasably secured within slot 41, and at least a portion of the right side 38 of securing member 25 is releasably secured within slot 43 (FIGS. 12-13). In exemplary embodiments, at least a portion of left side 36 extends through slot 41 and at least a portion of right side 38 extends through slot 43 after the end effectors 37, 39 are releasably secured to receiver member 17.

In this way, user-operable surgical device 19 is now releasably secured or attached to receiver member 17 of assembly 11, and a user may then move/position the assembly 11 to any desired position and/or location (e.g., for imaging/surgical purposes) by operating device 19 (e.g., either manually or tele-surgically). For example and as shown in FIG. 13, a user may then move and/or position the assembly 11 over, across and/or adjacent to at least a portion of tissue or organ 49 of a patient for imaging purposes.

As previously noted, the first end 31 of securing member 25 typically extends a greater distance beyond the longitudinal axis 35 of the post member 23 relative to the extension of the second end 33 of the securing member 25 beyond axis 35 (FIGS. 8 and 9), and the first end 27 of the post member 23 is typically wider than the second end 29 of the post member 23, and these structural features/configurations of receiver member 17 further ensure that surgical device 19 is appropriately releasably secured or attached to receiver member 17 (i.e., that end effectors 37, 39 are appropriately releasably secured or attached to the left and right sides 36, 38 of securing member 25). In other words and as depicted in FIGS. 9-13, since the user-operable surgical device 19 typically approaches the assembly 11 from the proximal 18 of the housing 14 (FIGS. 9-11), the configuration of having the second end 29 of the securing member 25 being not as laterally wide as the first end 31 allows the opened first and second end effectors 37, 39 (which are typically “V” shaped when opened) to quickly and easily be manipulated/positioned around the securing member 25 in order to ensure that surgical device 19 is appropriately releasably secured or attached to receiver member 17 (FIGS. 12-13).

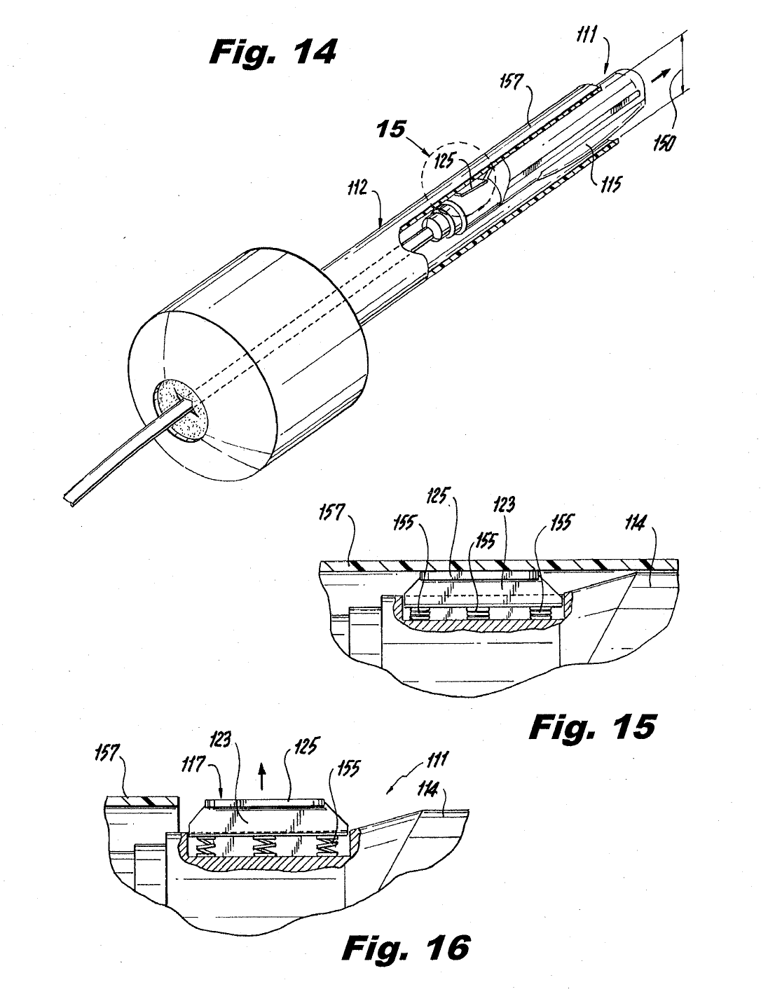

In an alternative embodiment and as shown in FIGS. 14-16, the receiver member 117 of assembly 111 is configured and dimensioned to be at least partially retractable within housing 114. For example, when the receiver member 117 is in the retracted position (e.g., at least partially within housing 114—FIGS. 14-15), this thereby allows the assembly 111 to be positioned in and/or inserted through a tool guide 112 having an inner diameter 150 that is substantially the same as or slightly larger than the greatest outer diameter of assembly 111. In other words, the retractable receiver member 117 allows assembly 111 to have a sleeker profile when inserted to the surgical site via tool guide 112 (FIGS. 14-15). Thus, after insertion of assembly 111 to the desired surgical site with the retracted receiver member 117, the post member 123 of receiver member 117 may then be un-retracted from housing 114 to then allow a surgical device 19 to be releasably secured to assembly 111 (e.g., to utilize imaging member 115 for imaging purposes, as similarly discussed above in relation to assembly 11).

In one embodiment and as depicted in FIGS. 14-15, post member 123 of receiver member 117 may be configured/dimensioned to be at least partially retractable within housing 114 via at least one spring member 155. When assembly 111 is inserted into tool guide 112, the inner wall 157 of tool guide 112 pushes against securing member 125, which thereby compresses spring members 155, which in turn allows at least a portion of post member 123 to be retracted within housing 114. It is noted that receiver member 117 and/or spring members 155 may be configured and dimensioned to allow the entire post member 123 (and securing member 125) to be retracted within housing 114 during insertion through tool guide 112. As shown in FIG. 16, when the securing member 125 is positioned out of the tool guide 112, the spring members 155 un-compress, thereby un-retracting the receiver member 117 from its position from inside the housing 114.

In another embodiment and as shown in FIGS. 19-20, post member 223 of receiver member 217 includes a movable sealing member 275, and post member 223 is configured/dimensioned to be at least partially retractable within fluidic chamber 259 of housing 214. In general, fluidic chamber 259 is a fluid-tight compartment (e.g., in conjunction with movable sealing member 275) that is configured and dimensioned to house and/or contain at least one fluid (e.g., when received from fluid line 261). In one embodiment, prior to inserting assembly 211 having receiver member 217 to the desired surgical (e.g. via tool guide 112), a user may force the post member 223 into the retracted position within the fluidic chamber 259 (FIGS. 14 and 19) (e.g., by pushing on securing member 225). Alternatively, it is noted that the inner wall 157 of tool guide 112 may push against securing member 225 to force at least a portion of post member 223 into the fluidic chamber 259. Fluid line 261 is typically in fluid communication with chamber 259 and with an actuating member 251 (e.g., an actuator). When it is desired to have the post member 223 positioned out of the fluidic chamber 259 (FIG. 20), a user may actuate the actuating member 251, which thereby forces fluid into the fluidic chamber 259 via the fluid line 261, which in turn forces post member 223 to its un-retracted position as shown in FIG. 20.

In exemplary embodiments, actuating member 251 is typically located or positioned outside of the body of the, patient. Alternatively, actuating member 251 may be positioned on housing 214 or some other location on assembly 211 (e.g., to be actuated via device 19). In one embodiment, post member 223 and securing member 225 are both substantially retracted or housed in housing 214 when the receiver member 217 is in the retracted position.

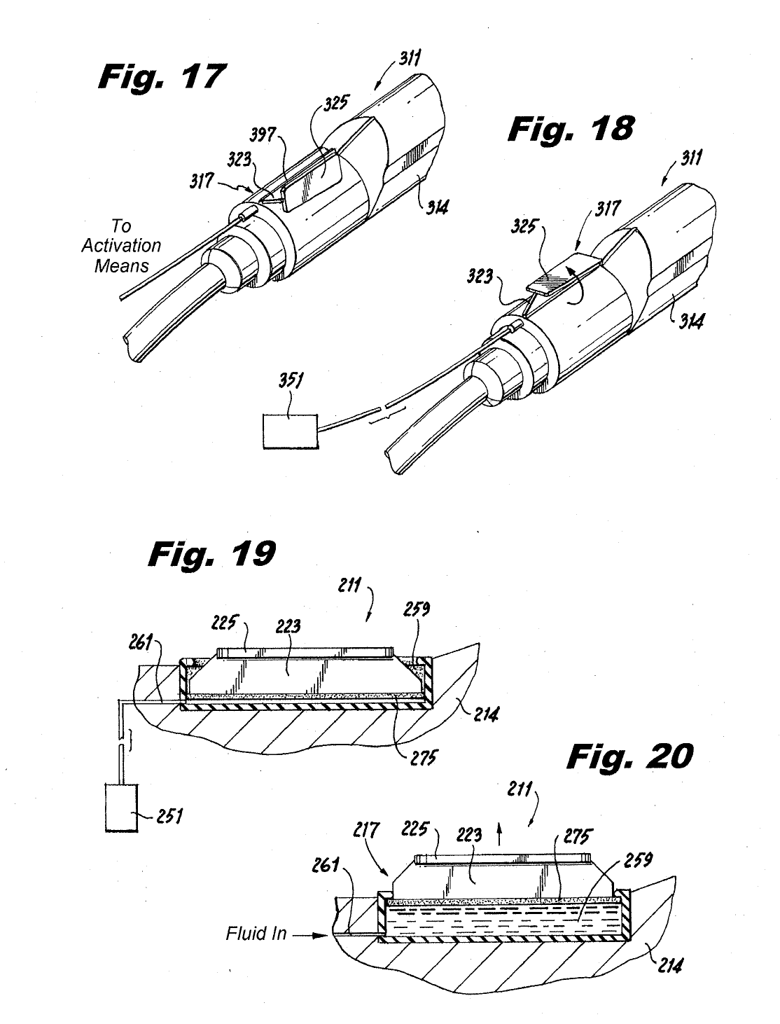

In another alternative embodiment and as shown in FIGS. 17-18, receiver member 317 (e.g., post member 323) may be configured and dimensioned to be at least partially foldable towards and/or relative to the surface of housing 314 (e.g., to allow assembly 311 to have a sleeker profile when inserted to the surgical site). Thus, after insertion of assembly 311 to the desired surgical site with the folded receiver member 317, the post member 323 of receiver member 317 may then be unfolded away from housing 314 to then allow a surgical device 19 to be releasably secured to assembly 311 as similarly discussed above in relation to assembly 11. Post member 323 may be folded or unfolded via actuating member 351, or manually (e.g., via device 19).

In one embodiment and as shown in FIGS. 17-18, receiver member 317 includes a hinge 397. Hinge 397 is configured and dimensioned to allow receiver member 317 (e.g., post member 323) to be at least partially foldable towards and/or relative to the surface of housing 314.

In one embodiment, prior to inserting assembly 311 having receiver member 317 to the desired surgical (e.g. via tool guide 112), a user may force the post member 323 into the folded position (FIG. 17) (e.g., by pushing on securing member 325). Alternatively, it is noted that the inner wall 157 of tool guide 112 may push against receiver member 317 to force the receiver member into the folded position (and the receiver member may thereby un-fold from the folded position after being positioned out of the tool guide via a spring of hinge 397, or via a user manually, or via actuating means 351, as discussed below).

When it is desired to have the post member 323 un-folded from the folded position, a user may actuate the actuating member 351, which actuates a biasing spring associated with the hinge 397 to force the post member 323 to the un-folded position as shown in FIG. 18.

Although the systems, assemblies and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

What is claimed is:

1. An assembly for use in a surgical procedure comprising:
a housing defining a substantially fin-shaped receiver member, the receiver member including a post member and a securing member;
wherein the post member extends from the housing and the securing member extends past both sides of the post member to define the substantially fin-shaped receiver member; and
wherein the substantially fin-shaped receiver member is configured and dimensioned to be releasably secured to a user-operable surgical device.

2. The assembly of claim 1 further comprising an imaging member mounted with respect to the housing.

3. The assembly of claim 2, wherein the imaging member is an ultrasound transducer.

4. The assembly of claim 1, wherein the receiver member is positioned at or near the proximal end of the housing.

5. The assembly of claim 1, wherein the user-operable surgical device includes first and second end effectors, the first end effector having a first slit and the second end effector having a second slit;
wherein the securing member has a first side and a second side; and
wherein at least a portion of the first side of the securing member extends through the first slit and at least a portion of the second side of the securing member extends through the second slit when the user-operable surgical device is releasable secured to the receiver member.

6. The assembly of claim 1, wherein the receiver member is integrally formed from the housing.

7. The assembly of claim 1, wherein the securing member has a first end and a second end and the post member defines a longitudinal axis; and
wherein the first end of the securing member extends a greater distance from the longitudinal axis relative to distance that the second end of the securing member extends from the longitudinal axis.

8. The assembly of claim 1, wherein the post member has a first end and a second end, the first end wider than the second end.

9. The assembly of claim 1, wherein at least a portion of the receiver member is retractable within the housing.

10. The assembly of claim 1, wherein at least a portion of the receiver member is foldable with respect to the housing.

11. The assembly of claim 1, wherein the housing is mounted with respect to a flexible cable.

12. The assembly of claim 1, wherein the user-operable surgical device is a minimally invasive user-operable surgical device.

13. The assembly of claim 2, wherein the imaging member is configured and dimensioned to capture an image of a surgical site.

14. The assembly of claim 1, wherein the housing and receiver member are configured and dimensioned to be: (i) inserted through a guide tool located in a minimally invasive incision of a patient, and (ii) moved to a surgical site within the patient.

15. A method for attaching an assembly to a surgical device comprising:
providing a housing defining a substantially fin-shaped receiver member, the receiver member including a post member and a securing member, with the post member extending from the housing and the securing member extending past both sides of the post member to define the substantially fin-shaped receiver member;
releasably securing a user-operable surgical device to the substantially fin-shaped receiver member.

16. The method of claim 15 further comprising mounting an imaging member with respect to the housing.

17. The method of claim 16, wherein the imaging member is an ultrasound transducer.

18. The method of claim 15, wherein the user-operable surgical device includes first and second end effectors, the first end effector having a first slit and the second end effector having a second slit;
wherein the securing member has a first side and a second side; and
wherein at least a portion of the first side of the securing member extends through the first slit and at least a portion of the second side of the securing member extends through the second slit when the user-operable surgical device is releasable secured to the receiver member.

19. The method of claim 15, wherein the securing member has a first end and a second end and the post member defines a longitudinal axis; and
wherein the first end of the securing member extends a greater distance from the longitudinal axis relative to distance that the second end of the securing member extends from the longitudinal axis.

20. The method of claim 15, wherein the post member has a first end and a second end, the first end wider than the second end.

21. The method of claim 15, wherein at least a portion of the receiver member is retractable within the housing.

22. An imaging assembly comprising:
a housing defining a substantially fin-shaped receiver member, the receiver member including a post member and a securing member;
an imaging member mounted with respect to the housing;
wherein post member extends from the housing and the securing member extends past both sides of the post member to define the substantially fin-shaped receiver member;
wherein the securing member has a first end and a second end and the post member defines a longitudinal axis;
wherein the first end of the securing member extends a greater distance from the longitudinal axis relative to distance that the second end of the securing member extends from the longitudinal axis;
wherein the receiver member is positioned at or near the proximal end of the housing;
wherein the substantially fin-shaped receiver member is configured and dimensioned to be releasably secured to a user-operable surgical device;
wherein the user-operable surgical device includes first and second end effectors, the first end effector having a first slit and the second end effector having a second slit;
wherein the securing member has a first side and a second side; and
wherein at least a portion of the first side of the securing member extends through the first slit and at least a portion of the second side of the securing member extends through the second slit when the user-operable surgical device is releasable secured to the receiver member.
